(19) United States
(12) Patent Application Publication
(10) Pub. No.: US 2017/0319396 A1
(43) Pub. Date: Nov. 9, 2017

(54) Feminine Hygiene Assembly

(71) Applicant: Shelley Martin, Westminster, CA (US)

(72) Inventor: Shelley Martin, Westminster, CA (US)

(21) Appl. No.: 15/146,057

(22) Filed: May 4, 2016

(51) Int. Cl. A61F 13/20; A61F 13/472

(52) U.S. Cl. CPC A61F 13/208; A61F 13/47236; A61F 13/202; A61F 13/204; A61F 2013/4729

(57) ABSTRACT

A feminine hygiene assembly includes a pad that may be positioned against a labia. The pad is comprised of a fluid absorbent material to absorb menstrual fluids. A plug is coupled to the pad. The plug may be substantially inserted into a vaginal opening when the pad is positioned against the labia. Thus, the pad is inhibited from sliding on the labia. The plug is comprised of a fluid absorbent material to wick the menstrual fluids into the pad.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIE THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

### (2) Description of Related Art Including Information Disclosed Under 37 CFR 1.97 and 1.98

The disclosure and prior art relates to hygiene devices and more particularly pertains to a new hygiene device for absorbing menstrual fluids.

### BRIEF SUMMARY OF THE INVENTION

An embodiment of the disclosure meets the needs presented above by generally comprising a pad that may be positioned against a labia. The pad is comprised of a fluid absorbent material to absorb menstrual fluids. A plug is coupled to the pad. The plug may be substantially inserted into a vaginal opening when the pad is positioned against the labia. Thus, the pad is inhibited from sliding on the labia. The plug is comprised of a fluid absorbent material to wick the menstrual fluids into the pad.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a top perspective view of a feminine hygiene assembly according to an embodiment of the disclosure.

FIG. 2 is a bottom view of an embodiment of the disclosure.

FIG. 3 is a right side view of an embodiment of the disclosure.

FIG. 4 is a top view of an embodiment of the disclosure.

FIG. 5 is a cross sectional view taken along line 5-5 of FIG. 4 of an embodiment of the disclosure.

FIG. 6 is a perspective in-use view of an embodiment of the disclosure.

### DETAILED DESCRIPTION OF THE INVENTION

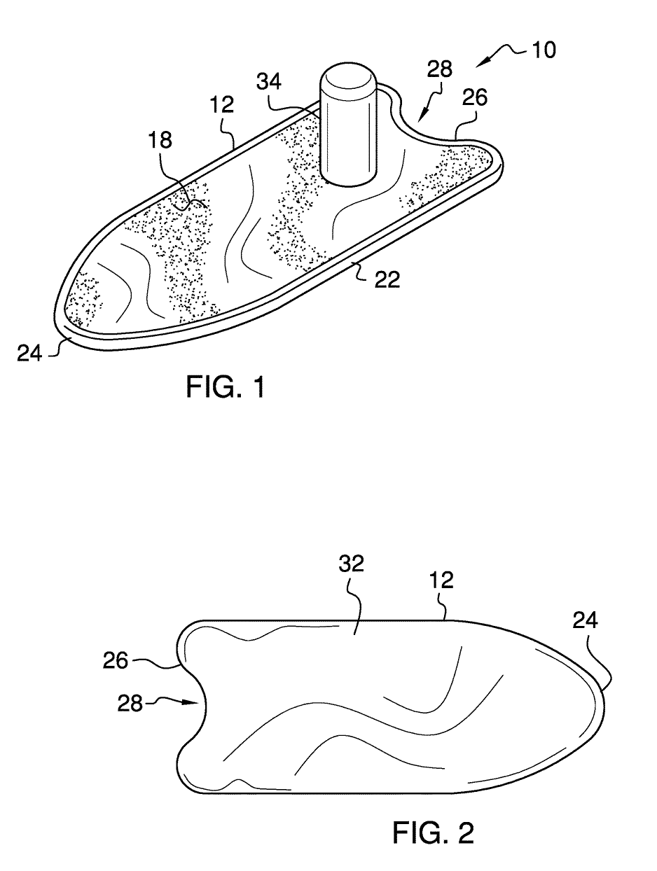

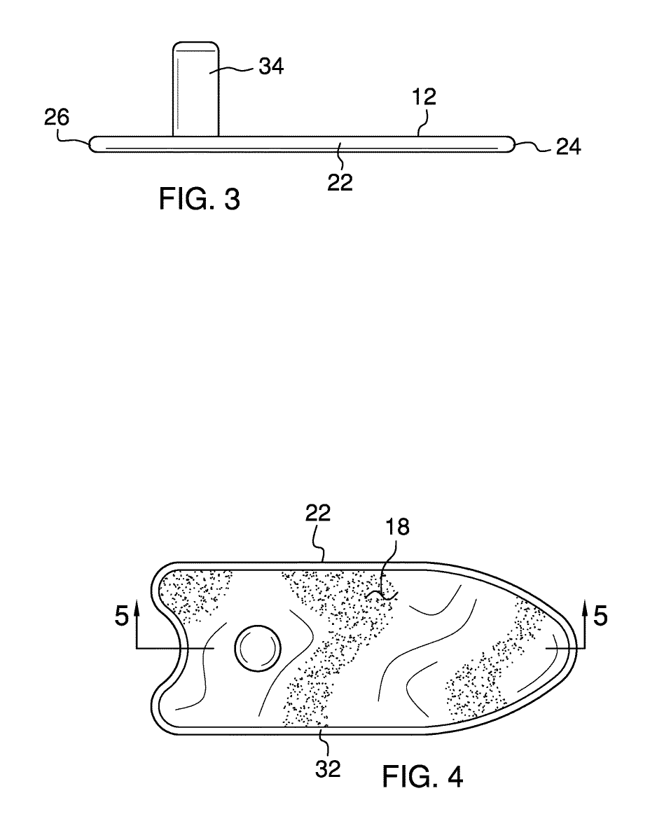

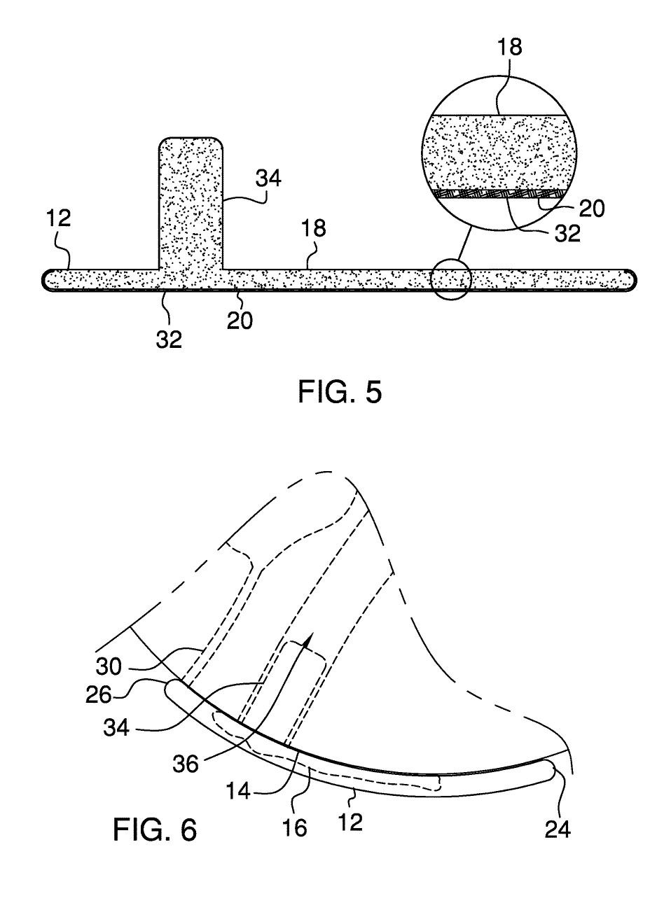

With reference now to the drawings, and in particular to FIGS. 1 through 6 thereof, a new hygiene device embodying the principles and concepts of an embodiment of the disclosure and generally designated by the reference numeral 10 will be described.

As best illustrated in FIGS. 1 through 6, the feminine hygiene assembly 10 generally comprises a pad 12 that may be positioned against a labia 14. The pad 12 is comprised of a fluid absorbent material to absorb menstrual fluids 16. The absorbent material may comprise a blend of cotton and rayon or the like. The pad 12 has a first surface 18, a second surface 20 and a peripheral edge 22 extending therebetween. The first surface 18 abuts the labia 14 when the pad 12 is positioned against the labia 14.

The peripheral edge 22 has a front side 24 and a back side 26. The pad 12 is elongated between the front side 24 and the back side 26. The back side 26 curves inwardly toward the front side 24 to define a urethra space 28. The front side 24 tapers to a point. The pad 12 is positioned having the urethra space 28 being aligned with a urethra 30. Thus, the pad 12 is inhibited from interfering with urination when the pad 12 is positioned against the labia 14.

A sheet 32 is provided. The sheet 32 is coupled to the second surface 20. The sheet 32 completely covers the second surface 20. The sheet 32 is comprised of a fluid impermeable material such as plastic or the like. Thus, the sheet 32 inhibits the menstrual fluids from passing through the sheet 32 and soiling clothing or the like. Moreover, the sheet 32 wraps upwardly along the peripheral edge 22 of the pad 12.

A plug 34 is provided. The plug 34 is coupled to the pad 12. The plug 34 may be substantially inserted into a vaginal opening 36 when the pad 12 is positioned against the labia 14. Thus, the pad 12 is inhibited from sliding on the labia 14.

The plug 34 is comprised of a fluid absorbent material. Thus, the plug 34 may wick the menstrual fluids 16 into the pad 12. The fluid absorbent material may be a blend of cotton and rayon or the like. Moreover, the plug 34 may be comprised of a hypo-allergenic material. Thus, the plug 34 inhibits a user from developing toxic shock from the plug 34. The plug 34 is positioned on the first surface 18 and the plug 34 is centrally positioned on the first surface 18. The plug 34 is positioned closer to the back side 26 than the front side 24.

In use, the plug 34 is substantially inserted into the vaginal opening 36 such that the first surface 18 of the pad 12 abuts the labia 14. The plug 34 aligns the urethra space 28 with the urethra 30 when the pad 12 is positioned to abut the labia 14. Thus, the pad 12 does not need to be removed to facilitate urination. The plug 34 wicks the menstrual fluids 16 into the pad 12 thereby enhancing absorbing the menstrual fluids 16. The sheet 32 inhibits the menstrual fluids 16 from contacting clothing or the like. The pad 12 is removed from the labia 14 when the pad 12 becomes soaked with the menstrual fluids 16 and the pad 12 is discarded.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A feminine hygiene assembly combining elements of a tampon and a maxi-pad, said assembly comprising:
a pad being configured to be positioned against a labia, said pad being comprised of a fluid absorbent material wherein said pad is configured to absorb menstrual fluids; and
a plug being coupled to said pad wherein said plug is configured to be substantially inserted into a vaginal opening when said pad is positioned against the labia thereby inhibiting said pad from sliding on the labia, said plug being comprised of a fluid absorbent material wherein said plug is configured to wick the menstrual fluids into said pad.

2. The assembly according to claim 1, wherein said pad has a first surface, a second surface and a peripheral edge extending therebetween, said first surface being configured to abut the labia, said peripheral edge having a front side and a back side, said pad being elongated between said front side and said back side, said back side curving inwardly toward said front side to define a urethra space, said front side tapering to a point, said pad being configured to be positioned having said urethra space being aligned with a urethra thereby inhibiting said pad from interfering with urination when said pad is positioned against the labia.

3. The assembly according to claim 2, further comprising a sheet being coupled to said second surface, said sheet completely covering said second surface, said sheet being comprised of a fluid impermeable material wherein said sheet is configured to inhibit the menstrual fluids from passing through said sheet.

4. The assembly according to claim 1, wherein:
said pad has a first surface, a front side and a back side; and
said plug is positioned on said first surface, said plug being centrally positioned on said first surface, said plug being positioned closer to said back side than said front side.

5. A feminine hygiene assembly combining elements of a tampon and a maxi-pad, said assembly comprising:
a pad being configured to be positioned against a labia, said pad being comprised of a fluid absorbent material wherein said pad is configured to absorb menstrual fluids, said pad having a first surface, a second surface and a peripheral edge extending therebetween, said first surface being configured to abut the labia, said peripheral edge having a front side and a back side, said pad being elongated between said front side and said back side, said back side curving inwardly toward said to define a urethra space, said front side tapering to a point, said pad being configured to be positioned having said urethra space being aligned with a urethra thereby inhibiting said pad from interfering with urination when said pad is positioned against the labia;
a sheet being coupled to said second surface, said sheet completely covering said second surfaced, said sheet being comprised of a fluid impermeable material wherein said sheet is configured to inhibit the menstrual fluids from passing through said sheet; and
a plug being coupled to said pad wherein said plug is configured to be substantially inserted into a vaginal opening when said pad is positioned against the labia thereby inhibiting said pad from sliding on the labia, said plug being comprised of a fluid absorbent material wherein said plug is configured to wick the menstrual fluids into said pad, said plug being positioned on said first surface, said plug being centrally positioned on said first surface, said plug being positioned closer to said back side than said front side.
